(19) United States
(12) Patent Application Publication
(10) Pub. No.: US 2010/0151065 A1
(43) Pub. Date: Jun. 17, 2010

(54) USE OF A COSMETIC COMPOSITION FOR COMBATING THE EFFECTS OF ELECTROMAGNETIC WAVES ON THE SKIN

(75) Inventor: Olivier Courtin, Boulogne Billancourt (FR)

Correspondence Address: ROTHWELL, FIGG, ERNST & MANBECK, P.C., 1425 K STREET, N.W., SUITE 800, WASHINGTON, DC, 20005, US

(73) Assignee: Laboratories Clarins, Neuilly sur Seine (FR)

(21) Appl. No.: 12/307,463

(22) PCT Filed: Jul. 24, 2006
(86) PCT No.: PCT/FR2006/001796

§ 371 Date: May 15, 2009

(30) Foreign Application Priority Data
Jul. 6, 2006 (FR) 0606144

(51) Int. Cl. A61K 8/97; A61K 8/99; A61Q 19/00; C07C 69/24; C07K 2/00; A01H 5/00; C12N 1/20

(52) U.S. Cl. 424/769; 424/773; 424/780; 560/129; 530/350; 800/298; 435/252.1

(57) ABSTRACT

The invention relates to the cosmetic use of at least one epidermal differentiation activator for combating the effects of electromagnetic waves on the skin. The invention also relates to a cosmetic treatment process comprising the application, to the skin or the integuments, of a cosmetic composition comprising at least one epidermal differentiation activator for combating the effects of electromagnetic waves on the skin.

The present invention relates to the use of a cosmetic composition for combating the effects of electromagnetic waves on the skin and integuments.

The skin has a surface area of around 2 m² and is the largest organ of the body. Essential to life, a veritable interface between the body and the environment, it is an interactive barrier. It has great abilities to adapt or react in the face of attack. It is also a favored site for absorption of pollutants from the environment, with passage of substances which may have a direct cutaneous toxic effect.

Pollution may take on various appearances: besides the well-documented particulate and gaseous pollution a new form of pollution via electromagnetic waves is also added. Pollution is not limited to the outside, it also concerns the inside of places: the use of synthetic materials and/or household chemicals generates new pollutants. The main polluting agents are particulates and ozone that generate free radicals.

The skin is in direct contact with the polluting agents. Dermatologists are confronted, in urban zones, by an upsurge in irritated and sensitive skins and by a worsening of certain dermatoses.

Many studies have shown the irritant potential of polluting agents when they are in contact with the skin. They have an action on the modification of the composition of lipids of the skin and acidify its pH. These various modifications lead to a disruption in the barrier function of the skin and induce reactions of oxidative and inflammatory nature in the living layers of the epidermis with expression of stress proteins, proinflammatory cytokines and metalloproteinases.

Electromagnetic waves are a form of energy composed of vibrations of electric and magnetic fields. They cannot be seen or felt, but they are increasingly present in our domestic environment.

In our dwellings, we are exposed to magnetic fields generated by domestic electrical appliances and installations (microwave oven, television, radio, cell phone, etc.).

Starting from a relatively high threshold, depending on individual sensitivities, these electromagnetic fields may influence our metabolism.

The consequences of electromagnetic waves on the skin are less known. To evaluate their effects on a cutaneous level, the Applicant has developed an original model that makes it possible to expose an epidermis reconstructed in vitro to electromagnetic waves of a given frequency. The work of the Applicant has made it possible to highlight an increase in the production of heat shock proteins in response to stress, a drop in the synthesis of β-defensin II, a drop in the synthesis of filaggrin and of loricrin, and therefore mainly a drop in epidermal differentiation.

Terminal differentiation of the epidermis is a vectorized process, during which the keratinocyte—from the germinative basal layer of the epithelium—undergoes progressive metabolic and structural rearrangements that finally transform it to corneocyte. The stack of these corneocytes constitutes the horny layer, the outermost part of the epidermis that provides—through its great strength—the mechanical protection of the body and constitutes a barrier between the individual and his environment by opposing water losses and the penetration of exogenous molecules. The horny layer thus formed, strong and impermeable, makes it possible to limit the imperceptible loss of water and therefore to give the epidermis a good hydration of its upper layers giving the skin the suppleness and elasticity of skin qualified as “normal skin”.

A drop in epidermal differentiation leads to a poor structure of the stratum corneum. The result of this disturbed structure is a reduced cohesion of the cutaneous barrier, which becoming less impermeable, protects the skin less effectively from external attacks. It is followed by a drop in cutaneous hydration, a drop in the suppleness and elasticity of the skin.

The effects of waves on the epidermal model are expressed by a drop in filaggrin and loricrin. The marker used to monitor the effect of the drop in epidermal differentiation is loricrin. This is because this protein is the major constituent, at 66%, of the membrane of corneocytes (The Journal of Biological Chemistry, Vol. 270, No. 30, p. 17702-17711, 1995). The result of a drop in loricrin in the stratum corneum is a significant disorganization of its structure and therefore a poor cohesion of the epidermal layer.

The present invention therefore relates to the cosmetic use of at least one activator of epidermal differentiation for combating the effects of electromagnetic waves on the skin.

The expression “activator of epidermal differentiation” is understood to mean an active agent that makes it possible to significantly increase the epidermal differentiation relative to the control according to the technique of immunolabeling cytokeratin and filaggrin more precisely described in the experimental section. The effect of an active agent on the synthesis of cytokeratin 10 and on filaggrin, representing epidermal differentiation markers, is analyzed in cultures of human keratinocytes in a monolayer. The two markers of differentiation are revealed by immunocytochemistry. The intensity of the fluorescence makes it possible to locate the presence or absence of the markers.

The activator of epidermal differentiation according to the invention is preferably of plant or marine origin. Among the known activators of epidermal differentiation, mention may be made of: an extract of Salicornia herbacea, such as for example that sold by Codif under the name “huile de Salicorne” [samphire oil], a peptide extract of hazelnut, such as for example that sold by Solabia under the name “Nutéline C”, or caprylyl butyrate. Preferably, an extract of Rhodiola rosea, such as for example that sold by Arch Personal Care under the name “Rhodiola rosea extract”, an extract of Thermus thermophilus, such as for example that sold by Sederma under the name “Vénucéane” and an extract of Cedrelopsis grevei, such as for example that sold by Bayer Healthcare under the name “extract of Cedrelopsis grevei”, is chosen.

Rhodiola rosea is a perennial plant native to the high altitudes of the Arctic regions of Eastern Siberia: it grows on a cold and dry rocky sandy soil. It has a great ability to withstand chemical, biological or physical stresses. This plant is rich in phenolic compounds which have a high antioxidant power but also in flavonoids, salidroside, rosavin, rosin and rosarin.

Advantageously, the extract that can be used in the context of the present invention is derived from the roots of the plant and, preferably, it is a hydroglycolic extract. After drying, the Rhodiola rosea roots are macerated in a mixture of solvents, water and pentylene glycol and heated at a temperature of around 50° C. Adequate filtration finalizes the extract which is in the form of a liquid of amber color and of characteristic odor. It has the following analytical characteristics:
pH: 4.0-6.0; and
percentage of active material: 0.6-1.2%.

Advantageously, the extract that can be used in the context of the present invention may also be an extract obtained by microculturing a marine organism: Thermus thermophilus. This microorganism lives in the ocean depths at a depth of 2000 m, in the Guayamas Basin in the Gulf of California, at 75° C., at a pressure of 200 bar and in high concentrations of sulfur and heavy metals: extreme temperature conditions and a very pro-oxidant environment.

Advantageously, the extract that can be used in the context of the present invention is obtained by biotechnology. Thermus thermophilus bacteria are cultured on a standard fermentation medium, the filtrate thus obtained is concentrated by tangential ultrafiltration and finally filtered. The extract is in the form of a clear to slightly opalescent yellow-brown liquid of characteristic odor. It has the following characteristics:
pH: 7.0-8.0;
density: 1.010-1.030;
refractive index: 1.335-1.355.

Finally, advantageously, the extract that can be used in the context of the present invention may also be an extract of Cedrelopsis grevei. Cedrelopsis grevei is a to 22 m tree endemic to Madagascar and from the family of Ptaeroxylaceae. It is located especially in the dense and dry forests at an altitude of between 100 and 800 m.

Advantageously, the Cedrelopsis grevei extract used according to the invention is an extract of bark from the tree, and preferably it is a hydroglycolic or hydroglycerine extract. After having been dried, the ground bark is macerated in a mixture of alcohol and water. After evaporation of the alcohol and readjustment with butylene glycol, the extract is sterile filtered. The Cedrelopsis grevei extract is in the form of an amber liquid of characteristic odor.

The composition according to the invention may contain one or more plant or marine extracts that are activators of epidermal differentiation. More particularly, the composition according to the invention may contain one or more activators chosen from an extract of Salicornia herbacea, a peptide extract of hazelnut or caprylyl butyrate. And most preferably, the composition according to the invention may contain one or more extracts chosen from an extract of Rhodiola rosea, an extract of Thermus thermophilus and an extract of Cedrelopsis grevei.

The composition according to the invention contains:
around 0.01 to 10% by weight, and preferably 0.01 to 5% by weight of Rhodiola rosea extract; and/or
around 0.01 to 10% by weight, preferably 0.01 to 5% by weight of Thermus thermophilus extract; and/or
around 0.01 to 10% by weight, preferably 0.01 to 5% by weight of Cedrelopsis grevei extract.

The cosmetic composition of the present invention for topical application may constitute, in particular, a composition for cosmetic or dermatological protection, treatment or care for the face, for the neck, for the hands or for the body, such as for example day creams, night creams, suntan creams or oils, body milks, a hair composition (for example a hair lotion), a makeup composition (for example a foundation) or a self-tanning composition. The cosmetic composition of the present invention is, in one preferred usage form, a day care cream.

The cosmetic composition according to the present invention may contain one or more other components known to the person skilled in the art, such as formulation agents or additives known to be conventionally used in cosmetic compositions. By way of example and nonlimitingly, such formulation agents and additives may be hydrophilic or lipophilic gelling agents, softeners, dyes, solubilizing agents, texturing agents, fragrances, fillers, odor absorbers, film-forming active agents, preservatives, surfactants, emulsifiers, oils, glycols, vitamins, sunscreens, etc. By virtue of his knowledge in terms of cosmetics, a person skilled in the art will know which formulation agents to add to the cosmetic composition according to the invention and in what amounts depending on the desired properties.

Furthermore, the cosmetic composition according to the present invention may be in any form known to a person skilled in the art in the cosmetics field without any specific pharmaceutical restriction other than that for application to the skin. Thus, the cosmetic composition according to the invention may be in the form of an aqueous or alcoholic solution or suspension or of an oily suspension or of a solution or of a dispersion of lotion or serum type, of an emulsion with a liquid or semi-liquid consistency of milk type, obtained by dispersion of a fatty phase in an aqueous phase (oil-in-water emulsion: O/W) or conversely (water-in-oil: W/O), or of an emulsion of the O/W or W/O cream type, or of a gel, of a lotion or of a mask. The cosmetic formulations according to the invention can also be envisioned in the form of a mousse or else in the form of aerosol compositions also comprising a pressurized propellant.

The present invention also relates to a cosmetic treatment method comprising the application, to the skin or integuments, of a cosmetic composition comprising at least one activator of epidermal differentiation to combat the effects of electromagnetic waves on the skin. Said activator is preferably chosen from an extract of Rhodiola rosea, an extract of Thermus thermophilus and an extract of Cedrelopsis grevei.

The examples below relate, on the one hand, to the evaluation of an epidermal pro-differentiation activity and, on the other hand, to the evaluation of the effect of electromagnetic waves on the skin, and also to the evaluation of the protection provided by the use of an extract of Rhodiola rosea and of Thermus thermophilus. They also relate to the compositions that are the subject of the present invention.

The examples refer to the following figures, in which:

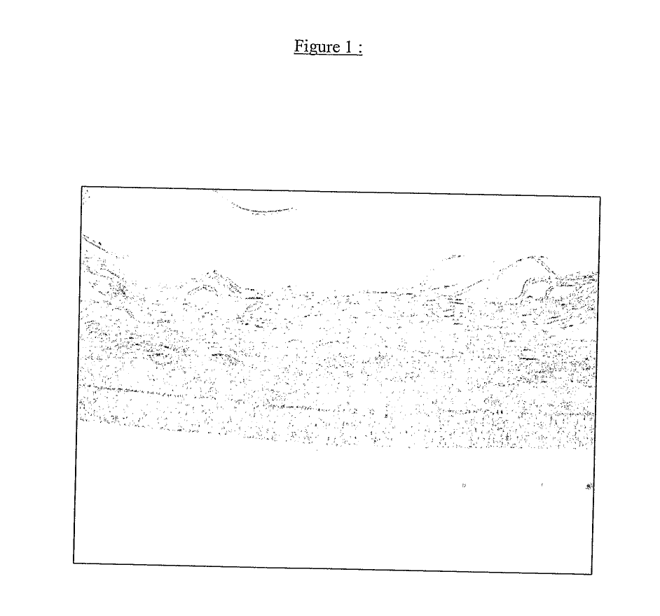

FIG. 1 represents a photo of the unexposed control, a control without anti-loricrin.

FIG. 2 represents a photo of the unexposed control, with loricrin labeling.

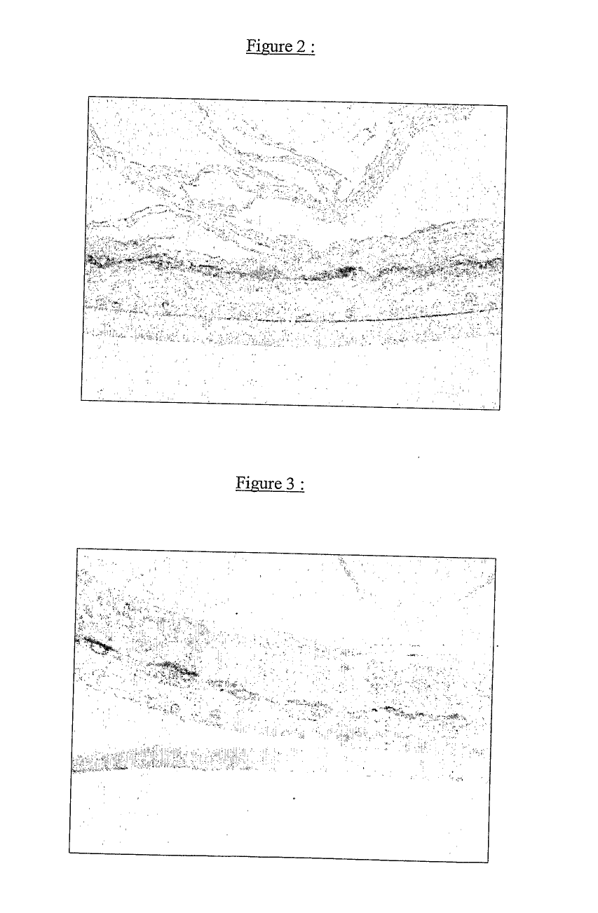

FIG. 3 represents a photo of the control exposed to the waves with loricrin labeling 18 hours after exposure.

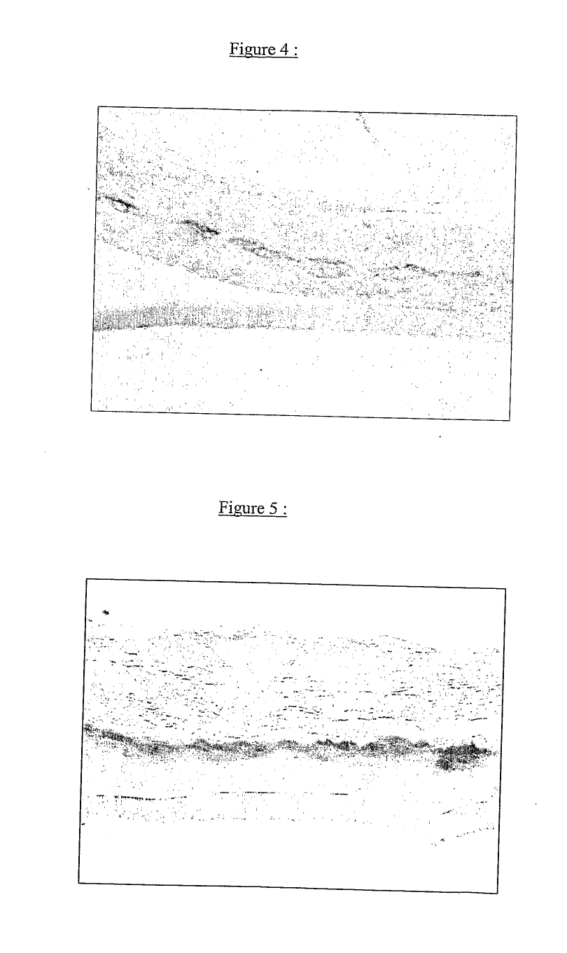

FIG. 4 represents the control 18 hours after exposure.

FIG. 5 represents the epidermis treated with the mixture of Thermus thermophilus extract and Rhodiola rosea extract and exposed to the waves, with loricrin labeling 18 hours after exposure.

### I. EVALUATION OF AN EPIDERMAL PRO-DIFFERENTIATION ACTIVITY

The protocol makes it possible to analyze the effect of an active agent on the synthesis of cytokeratin 10 and filaggrin, representing markers of epidermal differentiation, in cultures of human keratinocytes in a monolayer.

After treatments in nutrient media for 4 days, the two markers of differentiation are revealed by immunocytochemistry.

### A. Material and Methods

### 1. Cells Used

Type: human epidermal keratinocytes
Culture: 37° C., 5% CO₂
Medium: DMEM (Dulbecco Modified Essential Medium) medium+10% FVS (fetal veal serum)

### 2. Protocol

The cells are rinsed and incubated for 24 hours in a DMEM medium containing 2 mM of L-glutamine and 10% of FVS, nutrient medium used for the study. The cells are rinsed and incubated for 96 hours with the active agent to be tested at the various concentrations chosen.

Each cell layer is then rinsed, fixed in methanol (−20° C.) before revealing filaggrin and cytokeratin 10 by immunofluorescence.

### B. Results

Observed in ultraviolet light, fluorescein attached to the markers fluoresces green and makes it possible to locate them.

### II. EVALUATION OF THE EFFECT OF ELECTROMAGNETIC WAVES HAVING A FREQUENCY OF 900 MHz ON RECONSTRUCTED EPIDERMISES

### A. Material and Methods

### 1. Exposure to Electromagnetic Waves

A reconstructed epidermis model was exposed over 6 hours to waves of 900 MHz in an insulated chamber in order not to have any electromagnetic wave other than that of the desired frequency. The waves of 900 MHz chosen for the experiment correspond to the most frequent waves from cellular telephones.

After exposure, the epidermises are incubated at 37° C. in order to evaluate the disturbances caused by the exposure. An epidermis is incubated for 18 hours: it is compared to an unexposed control epidermis.

To determine the effects of the electromagnetic waves having a frequency of 900 MHz on the in vitro reconstructed epidermis, the DNA chip technique was used. This recent technique makes it possible to determine the possible activation or repression of gene expression. The chip chosen makes it possible to monitor the expression of 600 major genes of the epidermis. Confirmation by the RT-PCR or reverse-transcription PCR technique was then carried out.

### 2. Samples Used

### a) Samples for DNA Chip and RT-PCR

T1 (6 h) and T2 (6 h): unexposed controls, withdrawn after 6 h (=6 h controls).
EXP1 (6 h) and EXP2 (6 h): exposed for 6 h and withdrawn immediately after (=6 h exposed).
T3 (6 h+18 h) and T4 (6 h+18 h): controls not exposed for 6 h and withdrawn after 18 h of additional incubation (=6 h+18 h controls).
EXP3 (6 h+18 h) and EXP4 (6 h+18 h): exposed for 6 h and withdrawn after 18 h of additional incubation (=6 h+18 h exposed).

### b) Samples for Immunohistochemistry

Batches of epidermises exposed for 6 h and withdrawn after 18 h of incubation.
Batch of unexposed epidermises (controls)
Batch of exposed epidermises (exposed controls)
Batch of epidermises treated with an extract of Rhodiola rosea and an extract of Thermus thermophilus and exposed.

### 3. Effects on Gene Expression—DNA Chip

The analysis of the gene expression by DNA chip was carried out on “Custom ATLAS BA600/1” membranes chosen for their importance in cutaneous physiology.

The methodology used is that recommended by Clontech (Palo Alto, USA, protocol No. PT3140-1, version No. PR0X591—http://www.clontech.com/clontech/techinfo/manuals/PDF/PT3140-1.pdf).

The extraction/purification of the RNA of each sample led to the isolation of adequate amounts of total RNA.

The solutions of total RNA, containing all of the RNAse OUT enzyme in order to inhibit possible RNases, were treated with DNAse I according to the procedure recommended by Ambion (Ref. 1906—manual version 0503 (http://www.ambion.com/techlib/prot/bp_{—}1906.pdf)), to eliminate any trace of DNA contaminating the RNA.

The quality of the RNA was then verified on agarose gel for verification of the quality, of the quantity and of the absence of DNA.

RNAs originating from samples that had undergone the same treatment were pooled.

The following step was the purification of the pools of messenger RNA (mRNA) via hybridization of the poly(A) ends of the mRNA to biotinylated oligo(dT) primers and selective capture on streptavidine beads, according to the AtlasPure (Clontech) protocol. The multiple DNA probes labeled at ³³P were produced by reverse transcription of the mRNA bonded to the beads of poly(dT), using a batch of specific sequence primers immobilized on the chips, in the presence of [α³³P]-dATP. This step used the reactants and the protocol recommended by Clontech. The labeled probes were purified by size exclusion chromatography, the quality and the equivalence of the labeled probes was evaluated by liquid scintillation counting.

Two series of two “Custom ATLAS BA600/1” membranes were pretreated then the cDNA immobilized on each membrane were hybridized (68° C., overnight) with the corresponding labeled probes; the filters were then washed extensively (68° C.) and placed in individual plastic bags for analysis.
Series No. 1: membrane No. 1—unexposed controls, withdrawn after 6 h=6 h controls.
Membrane No. 2—exposed for 6 h and withdrawn immediately after =6 h exposed.
Series No. 2: membrane No. 1—controls not exposed for 6 h and withdrawn after 18 h of additional incubation=6 h+18 h controls.

Membrane No. 2—exposed for 6 h and withdrawn after 18 h of additional incubation=6 h+18 h exposed.

The analysis took place by direct quantification of the radioactivity of the spots using a PhosphorImager Cyclone apparatus (Packard Instruments; 3 h, then 72 h of acquisition) and BD AtlasImage™ 2.7 software (BD Biosciences Clontech).

### 4. Effects on the Expression of Loricrin —RT-PCR

The confirmation of the expression of the chosen marker, loricrin, was evaluated by RT-Q-PCR.

### a) Reverse Transcription

The reverse transcription reaction of the mRNA was carried out in the presence of the oligo(dT) primer and the enzyme Superscript II (Gibco). This was followed by quantification, by fluorescence, of the cDNA synthesized and adjustment of the concentrations. A further quantification of each cDNA, after final dilution, was carried out before the PCR reaction.

### b) Quantitative PCR

PCR or PCA (polymerized chain amplification) reactions were carried out by quantitative PCR with the “Light Cycler” system (Roche Molecular Systems Inc.) and according to the procedures recommended by the supplier.

This system of analysis made it possible to carry out rapid and high-performance PCR reactions, by means of a prior perfecting of the analysis conditions of the various primers. It is formed from two main components:
a thermocycler: optimized due to the use of glass capillaries and to extremely rapid heat transfers; and
a fluorimeter: that makes it possible to continuously measure the intensity of fluorescence incorporated in the DNA (detection at 521 nm).

The reaction mixture (10 μl final) introduced into the capillaries for each sample is the following:
2.5 μl of cDNA diluted to 1/10^{th};
primers of the various markers used;
reaction mixture (Roche) containing the enzyme Taq DNA polymerase, the marker SYBR Green I (fluorophor that is inserted in double-stranded DNA, during the elongation step) and MgCl₂.

The PCR conditions are the following:
Activation: 10 min at 95° C.;
PCR reactions: [10 s at 95° C., 5 s at 64° C. and 35 s at 72° C.] 40 cycles; and
melting: 5 s at 95° C. then 5 s at 60° C.

### c) Analysis of the Q-PCR

The incorporation of fluorescence into the amplified DNA is measured continuously during the PCR cycles. This system makes it possible to obtain curves of the measurement of the fluorescence as a function of the PCR cycles and to thus evaluate a relative expression value for each marker.

The RE (relative expression) value is expressed in arbitrary units according to the following formula:

(1/2^{number of cycles})×10⁶

### 5. Effects on the Expression of Loricrin—Immunohistochemistry

Cross sections of the epidermises were taken using a microtome (thickness 5 μm, 2 slides per epidermis, several sections per slide) then kept at ambient temperature until the labeling was carried out.

The sections were deparaffinized in glacial acetone for 10 minutes at −20° C., then rinsed with distilled water. The immunohistochemical labeling was carried out using the Autostainer machine (DakoCytomation) and the LSAB+ kit (DakoCytomation K067911). In brief, the blades were treated with hydrogen peroxide in order to block endogenous peroxydases, rinsed, then incubated for 30 minutes at ambient temperature in the anti-loricrin primary antibody solution. After washings, the sections were incubated for 15 minutes at ambient temperature with the biotin-coupled secondary antibody solution, rinsed again then incubated for 15 minutes with the streptavidine-coupled peroxydase solution. The enzymatic revelation was carried out using the mixture of substrate/chromogen from the LSAB+ kit. The slides were then washed, counterstained with a solution of hematoxylin (DakoCytomation S3309), rinsed with distilled water then mounted in a Glycergel aqueous medium (DakoCytomation C0563).

The sections were observed using a NIKON Diaphot 300 inverted microscope (40× lens). The images were captured using a COHU camera controlled by Lucia 7.0 software.

### B. Results

### 1. Expression of the Loricrin Gene —DNA Chip

The results are expressed in relative expression (RE) units. These levels are corrected for the average background noise present in each membrane and for differences in labeling intensity of the various probes used (this correction is carried out on the basis of the differences in labeling intensity of the reference genes).

In this experiment, the exposure of the epidermises to the waves reduced the expression level of loricrin by 38% 2 hours after exposure and by 28% 18 hours after exposure. These variations, of low intensity, needed to be verified by quantitative RT-PCR.

### 2. Expression of the Loricrin Gene —RT-PCR

The quantitative analysis of the degree of mRNA corresponding to the loricrin was carried out by quantitative RT-PCR. The exposure of the epidermises to the waves reduced the expression level of the loricrin by 20% 2 hours after exposure and increased it by 20% 18 hours after exposure.

### 3. Expression of the Loricrin Gene—Immunohistochemistry

### a) Secondary Antibody Control Alone (FIG. 1)

As expected, no signal was observed within the epidermis when the labeling was carried out in the presence of the secondary antibody alone (without the anti-loricrin primary antibody).

### b) Effect of the Exposure to the Waves (FIGS. 2 and 3)

The anti-loricrin labeling was mainly located in the granular layer. The labeling was less intense and discontinuous in the epidermises exposed to telephone waves.

c) Effect of the Rhodiola rosea Extract and of the Thermus thermophilus Extract (FIGS. 4 and 5)

Under these experimental conditions, the treatment of the epidermises exposed to the waves using the product “Active agent A+B” increased the labeling intensity relative to the exposed control. The labeling might be greater than that of the unexposed control.

### C. Conclusion

The DNA chip technique is powerful for detecting the effects of treatments on the expression level of many genes in a single test, it has therefore made it possible for us to carry out a first screening. It has, on the other hand, the drawback of not being very quantitative and of only being reliable for large expression variation amplitudes. Conversely, the quantitative RT-PCR technique has the advantage of measuring the level of messenger RNA of a given gene with precision. In the tests carried out, the analysis by DNA chip indicated a reduction in the expression of loricrin after exposure of the epidermises to the waves, whereas RT-PCR showed a reduction of 20% in the expression level of loricrin 2 hours after exposure and an increase of 20% 18 hours after exposure. The analysis by immunolabeling of sections of epidermises confirmed a reduction of loricrin 18 hours after exposure. This apparent contradiction could be explained by the fact that the reduction in mRNA after 2 hours is only expressed at the protein level much later.

The extracts of Rhodiola rosea and of Thermus thermophilus showed a significant protection against this inhibitory effect of the waves on the expression of loricrin, and therefore epidermal differentiation.

### III. EXAMPLES

### A. Anti-Wave Cream Gel

| | % |
|---|---|
| DEMINERALIZED WATER | qs for 100 |
| PEMULEN TR1 | 0.5 |
| GLYCEROL | 5.0 |
| SEPIGEL 305 | 1.0 |
| EXTRACT OF RHODIOLA ROSEA | 2.0 |
| EXTRACT OF THERMUS THERMOPHILUS | 2.0 |
| ISONONYL ISONONANOATE | 7.0 |
| C12-C15 ALKYL BENZOATE | 3.0 |
| SILICONE OIL | 2.0 |
| SODIUM HYDROXIDE | 0.2 |
| FRAGRANCE | 0.3 |
| COLORANTS AT 1% | 0.12 |
| PRESERVATIVES | 1.0 |

### B. Anti-Wave Cream

| | % |
|---|---|
| DEMINERALIZED WATER | qs for 100 |
| CETEARYL GLUCOSIDE | 5.0 |
| C8-C10 TRIGLYCERIDES | 10 |
| SILICONE OIL | 2.0 |
| VOLATILE SILICONE | 5.0 |
| CARBOMER | 0.2 |
| GLYCEROL | 5.0 |
| EXTRACT OF RHODIOLA ROSEA | 2.0 |
| EXTRACT OF THERMUS THERMOPHILUS | 2.0 |
| FRAGRANCE | 0.3 |
| PRESERVATIVES | 1.0 |

### C. Anti-Wave Skin Lotion

| | % |
|---|---|
| DEMINERALIZED WATER | qs for 100 |
| DISODIUM EDTA | 0.2 |
| SODIUM CHLORIDE | 1.0 |
| EXTRACT OF RHODIOLA ROSEA | 2.0 |
| EXTRACT OF THERMUS THERMOPHILUS | 2.0 |
| EXTRACT OF CEDRELOPSIS GREVEI | 2.0 |
| ROSEMARY WATER | 4.0 |
| GLYCOL | 1.0 |
| PRESERVATIVES | 1.0 |

### D. Anti-Wave Hair Lotion

| | % |
|---|---|
| DIMETHICONE | 8.00 |
| COLORANT SOLUTION | 0.60 |
| ESSENTIAL OILS | 0.40 |
| NON-DENATURED 96.2° ALCOHOL | 25.00 |
| EXTRACT OF CEDRELOPSIS GREVEI | 2.00 |
| EXTRACT OF RHODIOLA ROSEA | 2.00 |
| EXTRACT OF THERMUS THERMOPHILUS | 2.00 |
| FRAGRANCE | 1.00 |
| DEMINERALIZED WATER | qs for 100 |

### E. Anti-Wave Foundation

| | % |
|---|---|
| EXTRACT OF CEDRELOPSIS GREVEI | 2.00 |
| EXTRACT OF RHODIOLA ROSEA | 2.00 |
| EXTRACT OF THERMUS THERMOPHILUS | 2.00 |
| SILICONE EMULSIFIER | 5.00 |
| COEMULSIFIER | 0.50 |
| SORBIC ACID | 0.05 |
| OCTYL METHOXYCINNAMATE | 5.00 |
| SILICONE WAX | 3.00 |
| VOLATILE SILICONE | 10.50 |
| BENTONE GEL | 2.00 |
| IRON OXIDES | 13.40 |
| SILICONE ELASTOMER | 2.00 |
| SILICONE POWDER | 0.90 |
| FRAGRANCE | 0.50 |
| PURE SODIUM CHLORIDE | 2.00 |
| EDTA | 0.10 |
| PRESERVATIVE | 1.00 |
| SERICITE | 0.20 |
| MICA/TITANIUM | 0.20 |
| PROPYLENE GLYCOL | 4.10 |
| DEMINERALIZED WATER | qs for 100 |

1. The use in a cosmetic composition of at least one activator of epidermal differentiation for combating the effects on the skin of electromagnetic waves generated by domestic electrical appliances and installations.

2. The use as claimed in claim 1, characterized in that said activator is an extract of plant or marine origin chosen from an extract of Rhodiola rosea, an extract of Thermus thermophilus, an extract of Cedrelopsis grevei, an extract of Salicornia herbacea, a peptide extract of hazelnut or caprylyl butyrate.

3. The use as claimed in claim 1, characterized in that said activator is chosen from an extract of Rhodiola rosea, an extract of Thermus thermophilus and an extract of Cedrelopsis grevei.

4. The use as claimed in claim 2, characterized in that the extract of Rhodiola rosea is an extract derived from the roots of the plant.

5. The use as claimed in claim 4, characterized in that the extract of Rhodiola rosea roots is a hydroglycolic extract.

6. The use as claimed in claim 2, characterized in that the extract of Thermus thermophilus is an extract of Thermus thermophilus bacteria obtained by biotechnology.

7. The use as claimed in claim 2, characterized in that the extract of Cedrelopsis grevei is an extract derived from the bark of the tree.

8. The use as claimed in claim 7, characterized in that the extract of Cedrelopsis grevei bark is a hydroglycolic or hydroglycerine extract.

9. The use as claimed in claim 2, characterized in that the composition contains:
around 0.01 to 10% by weight, and preferably 0.01 to 5% by weight of Rhodiola rosea extract; and/or
around 0.01 to 10% by weight, preferably 0.01 to 5% by weight of Thermus thermophilus extract; and/or
around 0.01 to 10% by weight, preferably 0.01 to 5% by weight of Cedrelopsis grevei extract.

10. The use as claimed in claim 1, for combating the effects of electromagnetic waves generated by cellular telephones.

11. The use as claimed in claim 1, for combating the effects of waves with a frequency of 900 MHz.

12. A cosmetic treatment method for combating the effects on the skin of electromagnetic waves generated by domestic electrical appliances and installations, comprising the application to the skin or the integuments of a cosmetic composition comprising at least one activator of epidermal differentiation.

13. The method as claimed in claim 12, characterized in that said activator is chosen from an extract of Rhodiola rosea, an extract of Thermus thermophilus and an extract of Cedrelopsis grevei.
